(19) United States
(12) Patent Application Publication
(10) Pub. No.: US 2022/0011090 A1
(43) Pub. Date: Jan. 13, 2022

(54) APPARATUS AND METHODS FOR DETECTING LIGHT

(71) Applicant: NOKIA TECHNOLOGIES OY, Espoo (FI)

(72) Inventors: Xin YUAN, New Providence, NJ (US); Paul WILFORD, Bernardsville, NJ (US)

(21) Appl. No.: 17/294,221

(22) PCT Filed: Nov. 13, 2019
(86) PCT No.: PCT/IB2019/059753

§ 371 Date: May 14, 2021

(30) Foreign Application Priority Data
Nov. 16, 2018 (EP) 18206841.1

(51) Int. Cl. G01B 9/02; G01J 3/45; G01J 3/28; G01J 3/02

(52) U.S. Cl. CPC G01B 9/02091; G01B 9/02044; A61B 3/102; G01J 3/2823; G01J 3/0229; G01J 3/45

(57) ABSTRACT

Apparatus and method for detecting light, the apparatus comprising: means for splitting an input beam of light, which is obtained from an optical coherence tomography arrangement into at least a first and a second beam of light; means for modulating the first beam of light to provide a first modulated beam of light and means for modulating the second beam of light to provide a second modulated beam of light; means for dispersing the first modulated beam of light to provide a first dispersed beam of light and means for dispersing the second modulated beam of light to provide a second dispersed beam of light; means for detecting the first dispersed beam of light and means for detecting the second dispersed beam of light, the means for detecting being configured to convert the detected beams of light into electrical output signals.

### CROSS-REFERENCE TO RELATED APPLICATIONS

This non-provisional patent application is a National Phase Entry under 35 U.S.C. § 371 of International Patent Application No. PCT/IB2019/059753 filed on Nov. 13, 2019 and entitled “Apparatus and Methods for Detecting Light,” which is incorporated by reference in its entirety as if fully provided herein.

### TECHNOLOGICAL FIELD

Examples of the disclosure relate to apparatus and methods for detecting light. In particular they relate to apparatus and methods for detecting light from an optical coherence tomography arrangement.

### BACKGROUND

Optical coherence tomography enables cross sectional imaging of an object such as a retina or other part of a body by detecting the light reflected from internal structures within the object.

It is useful to provide means for detecting the light from optical coherence tomography arrangements which enable a high quality image to be obtained.

### BRIEF SUMMARY

According to various, but not necessarily all, examples of the disclosure there is provided an apparatus comprising: means for splitting an input beam of light into at least a first beam of light and a second beam of light wherein the input beam of light is obtained from an optical coherence tomography arrangement and wherein the spectral bandwidth of the first beam of light and the spectral bandwidth of the second beam of light overlap; spatial modulation means for spatially modulating the first beam of light to provide a first spatially modulated beam of light and spatial modulation means for spatially modulating the second beam of light to provide a second spatially modulated beam of light; first dispersing means configured to disperse, in a first direction, the first spatially modulated beam of light to provide a first dispersed beam of light and second dispersing means configured to disperse, in a second direction different to the first direction, the second spatially modulated beam of light to provide a second dispersed beam of light; and means for detecting the first dispersed beam of light and means for detecting the second dispersed beam of light wherein the means for detecting the first dispersed beam of light and means for detecting the second dispersed beam of light are configured to convert the detected beams of light into electrical output signals.

The means for splitting an input beam of light into at least a first beam of light and a second beam of light may be configured to split the input beam of light into more than two beams of light.
The apparatus may comprise a first spatial modulation means for spatially modulating the first beam of light,
first dispersion means for dispersing the first spatially modulated beam of light and means for detecting the first dispersed beam of light, and
comprising:
a second spatial modulation means for spatially modulating the second beam of light, second dispersion means for dispersing the second spatially modulated beam of light and means for detecting the second dispersed beam of light.

The spatial modulation means for spatially modulating the beams of light comprise one or more coded apertures.

The one or more coded apertures may comprise a two dimensional pixelated coded aperture.

The means for modulating the beams of light may comprise at least a first portion having a first transparency to the beam of light and at least a second portion having a second transparency to the beam of light, the second transparency being different from the first transparency.

The first transparency and the second transparency may be wavelength dependent.

The first and second portions of the spatial modulation means for spatially modulating the input beam of light having different transparencies may be arranged in a random pattern.

The spatial modulation means for spatially modulating the beams of light may be arranged to convert a three dimensional signal into a two dimensional signal.

The spatial modulation means spatially for modulating the beams of light may be arranged to be moveable relative to the first and second dispersion means for dispersing the spatially modulated beams of light and means for detecting the dispersed beams of light.

The first dispersion means for dispersing the spatially modulated beams of light comprises at least one of: a prism or a grating, and wherein the second dispersion means for dispersing the spatially modulated beams of light comprises at least one of: a prism, and a grating.

The means for detecting the dispersed beams of light may comprise at least one of a: charge coupled device, and a complementary metal-oxide semiconductor sensor.

The means for detecting the dispersed beams of light may comprise a two dimensional array of sensors.

The optical coherence tomography arrangement may be arranged so that the input beam of light comprises different wavelengths of light and the different wavelengths of light provide information about different depths within the object.

According to various, but not necessarily all, examples of the disclosure there is provided an apparatus comprising: at least one beam splitter configured to split an input beam of light into at least a first beam of light and a second beam of light wherein the input beam of light is obtained from an optical coherence tomography arrangement; a first spatial modulator configured to spatially modulate the first beam of light to provide a first spatially modulated beam of light and a second spatial modulator configured to spatially modulate the second beam of light to provide a second spatially modulated beam of light; a first disperser configured to disperse the first spatially modulated beam of light to provide a first dispersed beam of light and a second disperser configured to disperse the second spatially modulated beam of light to provide a second dispersed beam of light; and a first detector configured to detect the first dispersed beam of light and a second detector configured to detect the second dispersed beam of light wherein the first detector the second detector are configured to convert the detected beams of light into electrical output signals. The spectral bandwidth of the first beam of light and the spectral bandwidth of the second beam of light overlap.

According to various, but not necessarily all, examples of the disclosure there is provided a method comprising: splitting an input beam of light into at least a first beam of light and a second beam of light wherein the input beam of light is obtained from an optical coherence tomography arrangement and wherein the spectral bandwidth of the first beam of light and the spectral bandwidth of the second beam of light overlap; spatially modulating the first beam of light to provide a first spatially modulated beam of light and spatially modulating the second beam of light to provide a second spatially modulated beam of light; dispersing the first spatially modulated beam of light, in a first direction, to provide a first dispersed beam of light and dispersing the second spatially modulated beam of light, in second direction different to the first direction, to provide a second dispersed beam of light; and detecting the first dispersed beam of light and detecting the second dispersed beam of light and converting the detected beams of light into electrical output signals.

The method may comprise splitting the input beam of light into more than two beams of light.

The method may comprise modulating, dispersing and detecting each of the beams of light.

The scope of protection sought for various embodiments of the invention is set out by the independent claims. The embodiments, examples and features, if any, described in this specification that do not fall under the scope of the independent claims are to be interpreted as examples useful for understanding various embodiments of the invention.

According to various, but not necessarily all, examples of the disclosure there is provided an apparatus for obtaining a three dimensional image of at least part of an object comprising:

a beam splitter means configured to split an input beam of light into at least a first beam of light and a second beam of light wherein the input beam of light is obtained from an optical coherence tomography arrangement;

a first spatial modulator configured to spatially modulate the first beam of light to provide a first spatially modulated beam of light;

a first disperser configured to disperse the first spatially modulated beam of light to provide a first dispersed beam of light;

a first detector configured to detect the first dispersed beam of light.

a second spatial modulator configured to spatially modulate the second beam of light to provide a second spatially modulated beam of light;

a second disperser configured to disperse the second spatially modulated beam of light to provide a second dispersed beam of light;

a second detector configured to detect the second first dispersed beam of light,

wherein the first detector the second detector are configured to convert the detected first dispersed beam of light and the detected second dispersed beam of light into electrical output signals; and

means for providing a three dimensional image of at least part of the object from the electrical output signals.

In some but not necessarily all examples, the first spatial modulator is configured to spatially modulate the first beam of light comprising light reflected from the object and the first spatial modulator comprises at least a first plurality of first portions having a first transparency to the first beam of light and at least a second plurality of second portions having a different transparency to the first beam of light, wherein the first and second portions of the first spatial modulator are pixelated and are arranged in a pixelated pattern.

In some but not necessarily all examples, the second spatial modulator is configured to spatially modulate the second beam of light comprising light reflected from the object and the second spatial modulator comprises at least a first plurality of first portions having a first transparency to the second beam of light and at least a second plurality of second portions having a different transparency to the second beam of light, wherein the first and second portions of the second spatial modulator are pixelated and are arranged in a pixelated pattern.

In some but not necessarily all examples, the first and second portions of the spatial modulator correspond to pixels in the detector.

According to various, but not necessarily all, examples of the disclosure there is provided apparatus comprising:

means for splitting an input beam of light into at least a first beam of light and a second beam of light wherein the input beam of light is obtained from an optical coherence tomography arrangement;

spatial modulation means for spatially modulating the first beam of light to provide a first spatially modulated beam of light and spatial modulation means for spatially modulating the second beam of light to provide a second spatially modulated beam of light;

means for dispersing the first spatially modulated beam of light to provide a first dispersed beam of light and means for dispersing the second spatially modulated beam of light to provide a second dispersed beam of light; and

means for detecting the first dispersed beam of light and means for detecting the second dispersed beam of light wherein the means for detecting the first dispersed beam of light and means for detecting the second dispersed beam of light are configured to convert the detected beams of light into electrical output signals.

The dispersing means for dispersing the first spatially modulated beam of light can be configured to disperse the first spatially modulated beam of light in a first direction to provide the first dispersed beam of light and the dispersing means for dispersing the second spatially modulated beam of light can be configured to disperse the second spatially modulated beam of light in a second direction, different to the first direction, to provide the second dispersed beam of light.

In some examples the first direction could be perpendicular, or substantially perpendicular, to the second direction.

### BRIEF DESCRIPTION OF THE DRAWINGS

Some example embodiments will now be described with reference to the accompanying drawings in which:

FIG. 1 illustrates an example apparatus;

FIG. 2 illustrates another example apparatus;

FIG. 3 illustrates an imaging principle;

FIG. 4 illustrates an optical coherence tomography arrangement and an apparatus;

FIG. 5 illustrates a method;

FIGS. 6A to 6H illustrate example images obtained using an example apparatus; and

FIG. 7 illustrates a method of using examples of the disclosure.

### DETAILED DESCRIPTION

Examples of the disclosure relate to an apparatus 101 which can be used as a detector for an optical coherence tomography (OCT) arrangement 121. The apparatus 101 provides for compressed sampling of the input beam of light and disperses different bandwidths of the beam of light. In examples of the disclosure the input beam of light is split into two or more beams of light. The two or more beams of lights can be modulated and dispersed independently of each other so as to provide a more detailed image. The image may be a high-quality image.

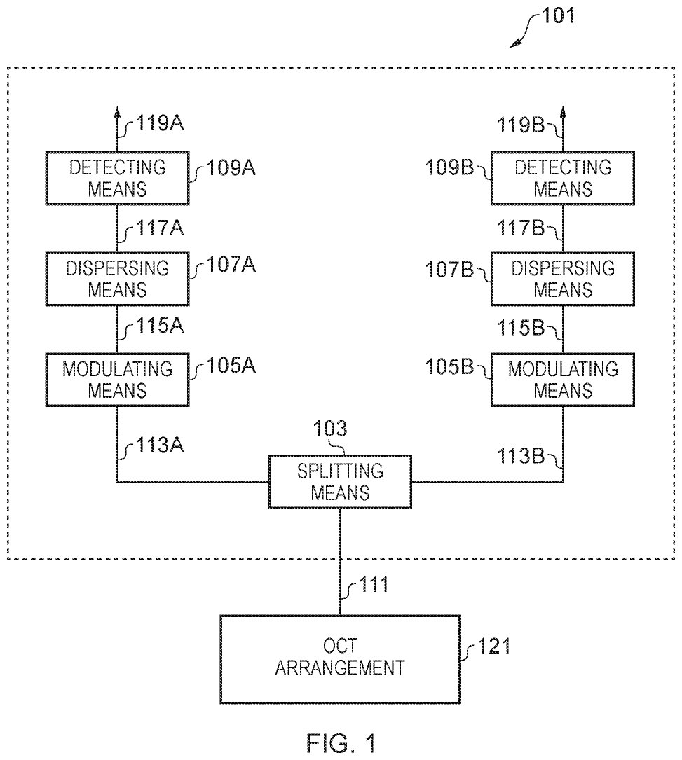

FIG. 1 schematically illustrates an example apparatus 101. The example apparatus 101 comprises means 103 for splitting an input beam of light 111 into at least a first beam of light 113A and a second beam of light 113B. The input beam of light 111 is obtained from an OCT arrangement 121. The OCT arrangement 121 may be a full-field spectral-domain OCT arrangement 121. The apparatus 101 also comprises means 105A for modulating the first beam of light 113A to provide a first modulated beam of light 115A and means 105B for modulating the second beam of light 113B to provide a second modulated beam of light 115B. The apparatus 101 also comprises means 107A for dispersing the first modulated beam of light 115A to provide a first dispersed beam of light 117A and means 107B for dispersing the second modulated beam of light 115B to provide a second dispersed beam of light 117B. The apparatus 101 also comprises means 109A for detecting the first dispersed beam of light 117A and means 109B for detecting the second dispersed beam of light 117B wherein the means 109A for detecting the first dispersed beam of light 117A and means 109B for detecting the second dispersed beam of light 117B are configured to convert the detected beams of light into electrical output signals 119A, 119B.

In at least some examples, processing means within or outside the apparatus 101 is configured to process the electrical output signal to provide an output, for example, a three dimensional image.

The means 103 for splitting an input beam of light 111 into at least a first beam of light 113A and a second beam of light 113B may comprise one or more beam splitters.

In some examples the one or more beam splitters may be configured as an optical beam splitter component that splits the input beam of light 111 so that each of the split beams 113A, 113B has the same frequency range. In some examples the one or more beam splitters may be configured to split the input beam of light 111 so that each of the split beams 113A, 113B has the same intensity. In other examples the one or more beam splitters may be configured to split the input beam of light 111 so that different split beams 113A, 113B have different intensities.

The one or more beam splitters are configured within the apparatus 101 so that when the apparatus 101 is coupled to an OCT arrangement 121, an input beam of light 111 from the OCT arrangement 121 is incident, at least in part, upon the one or more beam splitters 103.

In the example apparatus 101 shown in FIG. 1 the first beam of light 113A is provided to a first channel and the second beam of light 113B is provided to a second channel. Each of the first channel and the second channel enable sparse sampling of the respective beam of light. The first channel comprises first modulating means 105A, first dispersing means 107A and first detecting means 109A. The second channel comprises second modulating means 105B, second dispersing means 107B and second detecting means 109B. The two or more beams of lights 113A, 113B can thus be modulated and dispersed independently of each other.

The means 105A, 105B for modulating the first beam of light 113A and the second beam of light 113B comprise one or more modulators. The modulators are configured within the apparatus 101 so that the beams of light 113A, 113B from the one or more beam splitters are incident, at least in part on the one or more modulators. In the example shown in FIG. 1 the first beam of light 113A is incident on a first modulator and the second beam of light 113B is incident on a second modulator.

The means 105A for modulating can be means for spatially modulating (spatial modulation means) and can, in some examples, comprise one or more spatial modulators. An example of a spatial modulator is a spatially coded aperture. The means 105B for modulating can be means for spatially modulating (spatial modulation means) and can, in some examples, comprise one or more spatial modulators. An example of a spatial modulator is a spatially coded aperture.

The modulators enable compressed sampling of the beams of light 113A, 113B. The modulators are configured to selectively remove information from beams of light 113A, 113B so that only portions of the beams of light 113A, 113B are detected. In some examples the modulators may be configured to convert a three dimensional signal into a two dimensional signal.

The modulators may comprise any means which may be configured to spatially modulate the beams of light 113A, 113B. The spatial modulation occurs over a transverse cross-sectional area of the respective beams of light 113A, 113B. The modulation comprises amplitude modulation that varies in dependence upon a location within the transverse cross-sectional area of the respective beam of light 113A, 113B.

In some examples a modulator comprises a spatially coded aperture. The spatially coded aperture provides for spatial modulation over a cross-sectional area of the beam of light that passes through the coded aperture. The coded aperture is coded to provide amplitude modulation that varies in dependence upon a location within the aperture. The coded aperture defines a fixed two-dimensional pattern of spatially varying transparency. The spatially coded aperture physically modulates the beam of light to a spatially compressed/sparse format.

The spatially coded aperture may comprise a non-uniform optical mask or any other suitable type of aperture that provides amplitude modulation that varies in dependence upon a location within the aperture.

The spatially coded aperture may be a two-dimensional spatially coded aperture or any other suitable type of aperture. The two-dimensional spatially coded aperture defines a two-dimensional plane. The beam of light may travel in a direction normal (orthogonal) to the two-dimensional plane.

In other examples the modulators could comprise a liquid crystal on silicon (LCOS) modulator, a digital micro-mirror device (DMD), or any other suitable type of modulator.

The modulator can comprise multiple different portions that have a particular transparency. In some examples the modulators may comprise at least a first portion having a first level of transparency to the beams of light 113A, 113B and at least a second portion having a second, different level of transparency to the beams of light 113A, 113B. In some examples the modulator may comprise at least multiple spatially distributed non-overlapping first portions, that are distributed over an area in two dimensions and have a first level of transparency to the input beam of light 113A, 113B and at least multiple spatially distributed non-overlapping second portions that are distributed over the area in two dimensions and have a second, different level of transparency to the input beam of light 113A, 113B. In at least some examples, the spatially distributed first portions and the spatially distributed second portions do not overlap. The spatially distributed first portions and the spatially distributed second portions can be contiguous and, in some examples, the spatially distributed first portions and the spatially distributed second portions completely fill the area. The different levels of transparency may allow different levels of light to pass through the different portions of the modulators. It is to be appreciated that the modulators may comprise a plurality of first portions and a plurality of second portions. In some examples the modulators may be binary modulators so that only two different transparencies are provided by the respective portions of each modulator. In other examples the modulators could be a grey-scale aperture and may comprise more than two different levels of transparency in the different portions of the modulator.

In some examples the transparency of the different portions of the modulator may be wavelength dependent. In such examples the modulation of the beams of light 113A, 113B by the respective portions of the modulators will be dependent upon the wavelengths within the beams of light 113A, 113B.

The different portions of the one or more modulators may be arranged in any suitable pattern. In some examples the respective portions of the modulators having different transparencies are pixelated and arranged in a pixelated pattern. The pixelated pattern may comprise the respective portions of the modulators being arranged in an array of columns and rows of pixels. In some examples, the pixels are square or rectangular. The rows and columns may be parallel to, and in some examples correspond to, the pixels in the detecting means 109A, 109B.

The coded aperture can comprise multiple different portions that are coded with a particular transparency, for example, the coded aperture can be pixelated and comprise multiple different portions (pixels) that are arranged as an array in rows and columns, where the pixels are coded with a particular transparency. The two-dimensional pattern of pixels (portions) that have a first transparency is different to the two-dimensional pattern of pixels (portions) that have a second transparency, different to the first transparency.

The transparency at each pixel defines a fixed two-dimensional pattern of spatially varying transparency. In some examples, the transparency at each pixel in a row defines a fixed one-dimensional pattern of spatially varying transparency that does not repeat or does not repeat within a minimum number of columns. In some examples, the transparency at each pixel in a column defines a fixed one-dimensional pattern of spatially varying transparency that does not repeat or does not repeat within a minimum number of rows. In some examples, the transparency at each pixel defines a fixed two-dimensional pattern of spatially varying transparency that has a random or pseudorandom spatial distribution. In some examples, the pixels are coded as either opaque or transparent. In other examples, the pixels are coded using grey scale.

The size p of the pixels when projected onto a detector, can be directly proportional to a size d of pixels of the detector.

The number of transparent pixels, partially transparent pixels, and non-transparent (opaque) pixels in a spatially coded aperture may vary in different implementations of the disclosure. In some examples approximately half of the pixels of the modulator could be opaque so that half of the incident area of the modulator acts to block beams of light 113A, 113B while the other half allows the beams of light 113A, 113B, or partially pass through.

In some examples the different portions of the one or more modulators having different transparencies may be arranged in a random pattern (which encompasses pseudo random patterns) that is random in two dimensions. The random pattern may be an irregular pattern. The random pattern might not be defined or arranged in relation to any specific object. In some examples the different portions of the modulator may be arranged in a pseudo random pattern. In other examples the respective portions of the modulator may be arranged in a predetermined or customised pattern. The predetermined or customised pattern may be selected according to the object or type of object that is to be imaged by the OCT system.

In some examples the modulators may be fixed in position relative to the other components of the apparatus 101. In other examples the modulators may be arranged to be moveable relative to the other components of the apparatus 101. In particular the modulators may be moveable between imaging measurements so that the modulators can be shifted relative to the means 107A, 107B for dispersing the modulated beams of light 115A, 115B and the means 109A, 109B for detecting the dispersed beams of light 117A, 117B.

In the example shown in FIG. 1 the first modulator provides a first modulated beam of light 115A as an output and the second modulator provides a second modulated beam of light 115B as an output. The first modulator may be independent of the second modulator. In some examples the first modulator could be the same as the second modulator so that the same modulation is provided to the first beam of light 113A and the second beam of light 113B. In other examples the first modulator could be different to the second modulator so that different modulation is provided to the first beam of light 113A and the second beam of light 113B.

The example apparatus 101 shown in FIG. 1 comprises a first dispersing means 107A and a second dispersing means 107B. The first dispersing means 107A is configured within the apparatus 101 so that the first modulated beam of light 115A, or at least part of the first modulated beam of light 115A, provided by the first modulating means 105A is incident upon the first dispersing means 107A. The second dispersing means 107B is configured within the apparatus 101 so that the second modulated beam of light 115B, or at least part of the second modulated beam of light 115B, provided by the second modulating means 105B is incident upon the second dispersing means 107B.

The channels are separate. The first modulated beam of light 115A provided by the first modulating means 105A is not incident upon the second dispersing means 107B. The second modulated beam of light 115B is not incident upon the first dispersing means 107A.

The means 107A, 107B for dispersing the modulated beams of light 115A, 115B may comprise one or more dispersing elements. The dispersing elements may comprise any elements which cause different wavelengths of the modulated beams of light 115A, 115B to be refracted by different amounts. The one or more dispersing elements may comprise prisms, gratings or any other suitable elements.

In at least some examples, the first dispersing means 107A is configured to cause a wavelength dependent spatial shift of the same fixed spatially coded aperture, defined by the first modulator 105A. In at least some examples the spatial shift is only in the plane of the aperture/beam (2D dispersion). In at least some examples, the spatial shift is only in one dimension (1D dispersion). That one dimension can be aligned with a row (or a column) of pixels in the spatially coded aperture 105A and/or pixels of the first detector 109A. The second dispersing means 107B is configured to cause a wavelength dependent spatial shift of the same fixed spatially coded aperture, defined by the second modulator 105B. In at least some examples the spatial shift is only in the plane of the aperture/beam (2D dispersion). In at least some examples, the spatial shift is only in one dimension (1D dispersion). That one dimension can be aligned with a row (or a column) of pixels in the spatially coded aperture 105B and/or pixels of the second detector 109B.

The first dispersing means 107A may be configured to disperse the light in a first direction and the second dispersing means 107B may be configured to disperse the light in a second, different direction. In some examples the first direction could be perpendicular, or substantially perpendicular, to the second direction.

In some examples the first dispersing means 107A could be the same, or substantially the same, as the second dispersing means 107B so that the modulated beams of the light 115A, 115B are refracted by the same amounts or substantially the same amounts. In other examples the first dispersing means 107A and the second dispersing means 107B could be different so that the modulated beams of the light 115A, 115B are refracted by different amounts. In example apparatus 101 where the dispersing means 107A, 107B are different it may be necessary to perform processing on the detected signals to take into account the differences in the dispersing means 107A, 107B.

The first dispersing means 107A is configured to provide a first dispersed beam of light 117A as an output and the second dispersing means 107B is configured to provide a second dispersed beam of light 117B. The first means 109A for detecting the first dispersed beam of light 117A is configured within the apparatus 101 so that the first dispersed beam of light 117A, or at least part of the first dispersed beam of light 117A, is incident on the first means 109A for detecting the first dispersed beam of light 117A. The second means 109B for detecting the second dispersed beam of light 117B is configured within the apparatus 101 so that the second dispersed beam of light 117B, or at least part of the second dispersed beam of light 117B, is incident on the second means 109B for detecting the second dispersed beam of light 117B.

In this example, the channels are separate. The first dispersed beam of light 117A provided by the first dispersing means 107A is not incident upon the second detecting means 109B. The second dispersed beam of light 117B provided by the second dispersing means 107B is not incident upon the first detecting means 109A.

The means 109A, 109B for detecting the dispersed beams of light 117A, 117B comprise detectors. The detectors 109A, 109B may be arranged to transduce incident light into an electrical output signal. In some examples the detectors 109A, 109B may comprise charge-coupled devices, complementary metal-oxide semiconductor (CMOS) sensors or any other suitable type of sensors.

In some examples the detectors 109A, 109B may comprise two dimensional arrays of sensors. In other examples the detectors 109A, 109B may comprise linear detectors which may be scanned across a detecting plane. In some examples the first detector 109A and the second detector 109B could be the same type of detectors.

In the example apparatus 101 shown in FIG. 1 the first detector 109A provides a first output signal 119A and the second detector 109B provides a second output signal 119B. The first output signal 119A and the second output signal 119B both comprise information indicative of an object imaged by the OCT arrangement 121. However, the images that can be obtained from the output signals 119A, 119B may be different because the first dispersing means 107A and the second dispersing means 107B disperse the light in different directions. This means that different information could be comprised in the different output signals 119A, 119B.

In some examples the first output signal 119A and the second output signal 119B can be combined to provide a single output signal which represents an image of the object imaged by the OCT arrangement 121. This image obtained using two different modulating and dispersing channels may be more accurate and may comprise more information than an image obtained using a single channel. The image could be rendered on a display or other suitable user output device.

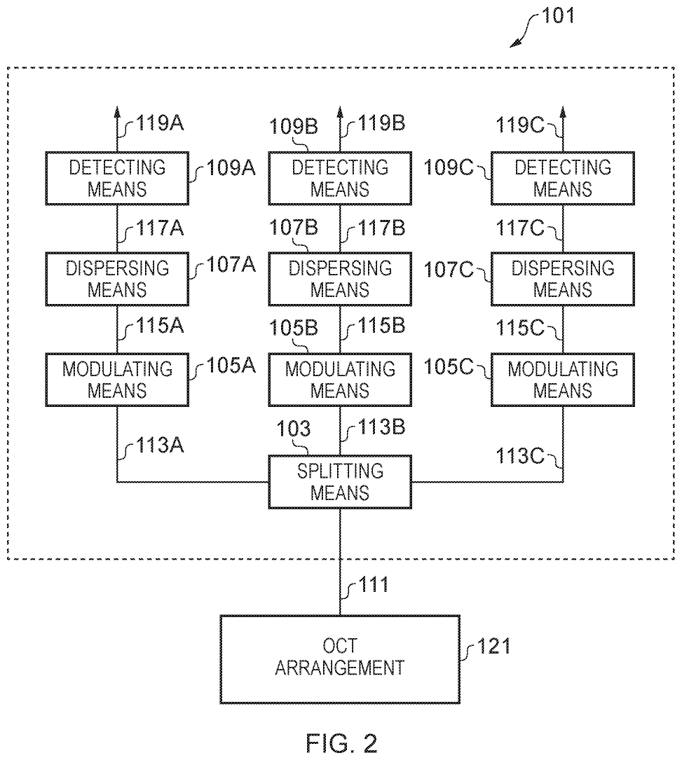

FIG. 2 illustrates another example apparatus 101 that could be provided in some examples of the disclosure. The example apparatus 101 shown in FIG. 2 is similar to the apparatus 101 shown in FIG. 1 except that the apparatus 101 shown in FIG. 2 comprises means 103 for splitting an input beam of light 111 into three beams of light directed to three independent channels for modulation, dispersion and detection. The apparatus 101 shown in FIG. 2 also comprises a plurality of means 105A, 105B, 105C for modulating respective beams of light 113A, 113B, 113C, a plurality of means 107A, 107B, 107C for dispersing respective modulated beams of light 115A, 115B, 115C and a plurality of means 109A, 109B, 109C for detecting respective dispersed beams of light 117A, 117B, 117C. The plurality of means 105A, 105B, 105C for modulating beams of light, plurality of means 107A, 107B, 107C for dispersing modulated beams of light and plurality of means 109A, 109B, 109C for detecting dispersed beams of light could be as described in relation to FIG. 1, corresponding reference numbers are used for corresponding features.

The splitting means 103 comprises any means which may be configured to split the input beam of light 111 into three separate beams of light. The separated beams of light can then be provided to three different channels. The splitting means 103 could comprise one or more beam splitters and/or any other suitable components. The three separated beams of light 113A, 113B, 113C can in at least some examples have the same frequency range.

In the example apparatus 101 shown in FIG. 2 the first beam of light 113A is provided to a first channel, the second beam of light 113B is provided to a second channel and the third beam of light 113C is provided to a third channel. Each of the first channel, the second channel and the third channel enable sparse sampling of the respective beam of light. Each of the first channel, the second channel and the third channel comprise modulating means 105A, 105B, 105C dispersing means 107A, 107B, 107C and detecting means 109A, 109B, 109C.

In the example apparatus 101 shown in FIG. 2 the first beam of light 113A from the splitting means 103 is provided to a first modulating means 105A to provide a first modulated beam of light 115A. The first modulated beam of light 115A is provided to a first dispersing means 107A to provide a first dispersed beam of light 117A. The first dispersed beam of light 117A is provided to the first detecting means 109A to provide a first output signal 119A. The second beam of light 113B from the splitting means 103 is provided to a second modulating means 105B to provide a second modulated beam of light 115B. The second modulated beam of light 115B is provided to a second dispersing means 107B to provide a second dispersed beam of light 117B. The second dispersed beam of light 117B is provided to the second detecting means 109B to provide a second output signal 119B. The third beam of light 113C from the splitting means 103 is provided to a third modulating means 105C to provide a third modulated beam of light 115C. The third modulated beam of light 115C is provided to a third dispersing means 107C to provide a third dispersed beam of light 117C. The third dispersed beam of light 117C is provided to the third detecting means 109C to provide a third output signal 119C.

The different dispersing means 107A, 107B, 107C in the different channels of the apparatus 101 may be configured to disperse the respective beams of light in different directions. In the example shown in FIG. 2 the first dispersing means 107A may be configured to disperse the light in a first direction, the second dispersing means 107B may be configured to disperse the light in a second, different direction. And the third dispersing means 107C may be configured to disperse the light in a third, different direction. In such examples the first direction could be at 60°, or substantially 60°, to the second direction and the second direction could be at 60°, or substantially 60°, to the third direction (120°, or substantially 120°, to the first direction).

In the example apparatus 101 shown in FIG. 2 the first detector 109A provides a first output signal 119A, the second detector 109B provides a second output signal 119B and the third detector 109C provides the third output signal 119C. The output signals 119A, 119B, 119C each comprise information indicative of an object imaged by the OCT arrangement 121. However, the images that can be obtained from the output signals 119A, 119B, 119C may be different because the dispersing means 107A, 107B, 107C disperse the light in different directions. This means that different information could be comprised in the different output signals 119A, 119B, 119C.

In some examples the three output signals 119A, 119B, 119C can be combined to provide a single output signal which represents an image of the object imaged by the OCT arrangement 121. This image obtained using three different modulating and dispersing channels may be more accurate and may comprise more information than an image obtained using a single channel or two channels. The image could be rendered on a display or other suitable user output device.

In the example shown in FIG. 2 the splitting means 103 is configured to split the input beam of light 111 into three separate beams of light 113A, 113B, 113C. In other examples the splitting means 103 could be configured to split the input beam of light 111 into more than three separate beams of light which could be provided to more than three separate channels. In examples of the disclosure the different channels could be configured to modulate, disperse and detect the respective beams of light independently of the other channels within the apparatus 101. The number of channels that are provided within the apparatus 101 may depend on factors such as signal to noise ratios, the objects being imaged and any other suitable factors.

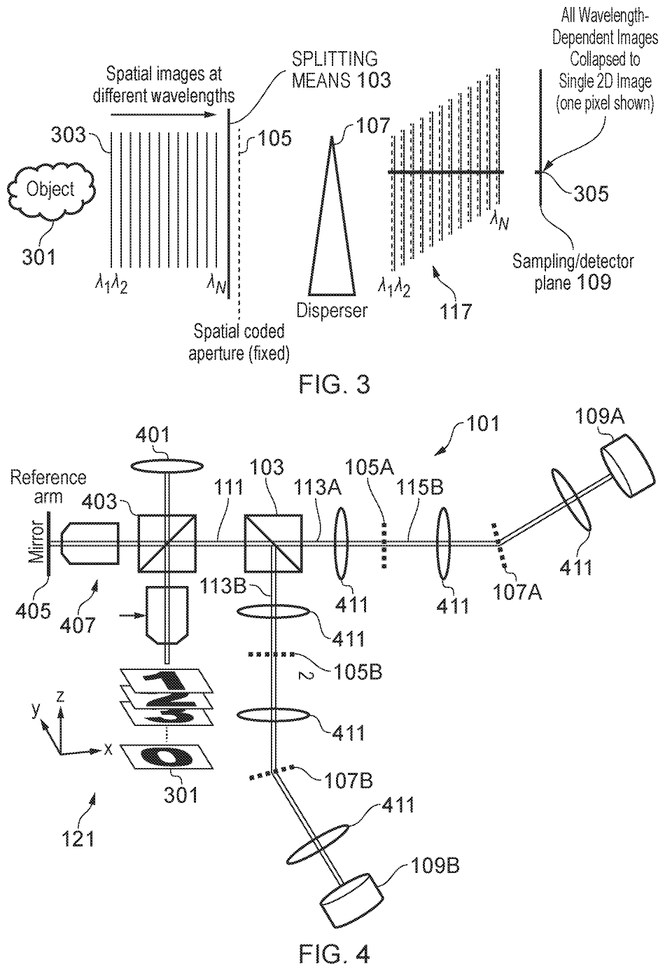

FIG. 3 illustrates an imaging principle of examples of the disclosure.

In the example of FIG. 3 an OCT arrangement (not shown for clarity) is used to image a three-dimensional object 301. The object 301 reflects broadband light which has been directed onto the object 301. The object 301 could be part of a subject's body such as a retina or any other suitable type of object 301.

Different wavelengths of the incident light are reflected differently depending upon the internal structure of the object 301. This provides a plurality of spatial images 303. Each of the spatial images 303 corresponds to a different wavelength of light λ₁ to λₙ. The different spatial images 303 therefore comprise information about the internal structure of the object 301. The different spatial images 303 may comprise a three dimensional signal.

The reflected beam of light is a three-dimensional data cube [x, y, λᵢ] with a two-dimensional slice [x,y], a spatial image 303, for each wavelength channel λᵢ.

In the example of FIG. 3 the spatial images 303 are provided to splitting means 103. The splitting means 103 is an optical component that splits the input beam of light 111 comprising the spatial images 303 into two or more broadband beams of light 113A, 113B that travel in different directions. Each of the respective beams of light 113A, 113B can then be provided from the beam splitter component to a different channel of an apparatus 101. Each beam of light is a three-dimensional data cube [x, y, λᵢ] with a two-dimensional slice [x,y], a spatial image 303, for each wavelength channel λᵢ.

Only one channel is shown in FIG. 3. It is to be appreciated that the imaging principle would be the same for the other channels of the apparatus 101.

In the example of FIG. 3 the modulating means 105 comprises a two dimensional coded aperture. Other types of modulating means 105 may be used in other examples of the disclosure, for example as previously described.

In the example of FIG. 2 the modulating means 105 is fixed in position relative to the dispersing means 107 and the detecting means 109. In other examples the modulating means 105 could be moveable relative to the dispersing means 107 and the detecting means 109 and any other suitable components of the apparatus 101.

The spatial images 303 in the beam of light 113 provided by the splitting means 103 are modulated by the coded aperture of the modulating means 105. The coded aperture blocks and/or at least partially blocks portions of each of the different spatial images 303. The coded aperture may be wavelength dependent so that different spatial images 303 corresponding to different wavelengths may be blocked by different amounts.

The spatial images 303 in the input beam of light are modulated by the spatially coded aperture 105 to produce a spatially modulated beam of light.

The spatially modulated beam of light is a sparse three-dimensional data cube [x, y, λ] with a two-dimensional slice [x,y] for each wavelength channel coded by the same fixed spatially coded aperture that has variable transparency in the x-y plane. The spatially modulated beam of light provided by the modulator 3 is then spread by the dispersing element 107.

The modulated beam of light 115 provided by the modulating means 105 is then spread by the dispersing means 107. In the example of FIG. 3 the dispersing means 107 comprises a prism. Other types of dispersing means 107 could be used in other examples of the disclosure. The dispersing means 107 refracts the modulated beam of light 115 to spatially spread the modulated beam of light 115. Different bandwidths of the spatial images 303 are spread by a different amount as shown schematically in FIG. 3. The distance by which a spatial image 303 is spread by the dispersing means 107 is dependent upon the wavelength of the spatial image 303.

The spatially modulated and dispersed beam of light 117A represents a skewed version of sparse three-dimensional data cube. The skew (offset), caused by the dispersing means 107, is within the x-y plane and is proportional to wavelength. In the example illustrated in FIG. 2 it is in the y-direction only. Each spatially coded two-dimensional slice [x,y] for each wavelength channel i is shifted (offset) yᵢ.

The spatially modulated and dispersed beam of light 117 is then incident upon the detecting means 109. The detecting means 109 comprises a plurality of pixels 305. Only one pixel 305 is shown for clarity in FIG. 3. The plurality of pixels 305 may be arranged in any suitable array. In the example of FIG. 3 the plurality of pixels 305 may be arranged in a matrix array comprising Nₓ rows (aligned with x direction) and N_{y} columns (aligned with y direction). Each pixel 305 detects the summation of the modulated and dispersed beam of light 117 for each of the different wavelengths λ₁ to λₙ for the area covered by the pixel 305.

As the different wavelengths λ₁ to λₙ in the dispersed beam of light 117 are shifted by different amounts the different wavelengths λ₁ to λₙ that are incident on a given pixel 305 of the detecting means 109 have passed though different portions of the modulating means 105. This means that the different wavelengths λ₁ to λₙ that are incident on a given pixel 305 of the detecting means 109 may be modulated by different amounts.

The detector 109 detects the superposition of the offset spatially coded two-dimensional slices [x,y] for each wavelength channel. This reduces the sparse three-dimensional data cube to a compressed two-dimensional projection in a single shot. It collapses overlapping differently masked spectrograms for different channels to a single spectrogram.

In the above examples the beam of light 113 that is provided from the splitting means 103 to the modulating means 105 can be represented as N_{λ} wavelength channels. Each of the wavelength channels has a spatial size Nₓ×N_{y}.

The signal provided to the detector 109 may be represented as Sₘ(x, y) where:

Sₘ(x, y)=∫_{λ}S₀(x, y, λ)M(x, y, λ)dλ.

The measurement z, of Sₘ(x, y), obtained by the (i,j)^{th} pixel where Z ∈ [symbol] ^{N}^{ₓ}^{×N}^{_{y}} is given by equation 1

z(i, j)=Σₙ_{_{λ}}₌₁^{N}^{_{λ}} S₀(i, j, n_{λ})M(i, j, n_{λ}). (1)

Where S₀(i, j, n_{λ}) is the three dimensional input signal and M(i, j, n_{λ}) is a function representing a combination of the modulating means 105 and the dispersing means 107. The value n_{λ} represents a spectral channel. The function M(i, j, n_{λ}) will be dependent on the transparencies of the portions on the modulating means 105, the spatial arrangement of the portions of the modulating means 105, the dispersing means 107 and any other suitable factors.

Therefore in an apparatus 101 comprising two channels the measurement z₁ obtained by the (i, j)^{th} pixel of the first detecting means 107A is given by equation 2 and the measurement z₂ obtained by the (i, j)^{th} pixel of the second detecting means 107B is given by equation 3

z₁(i, j)=Σₙ_{_{λ}}₌₁^{N}^{_{λ}} S₀(i, j, n_{λ})M₁(i, j, n_{λ}). (2)

z₂(i, j)=Σₙ_{_{λ}}₌₁^{N}^{_{λ}} S₀(i, j, n_{λ})M₂(i, j, n_{λ}). (3)

The function M(i, j, n_{λ}) can be modelled as a series of 2D masks for each wavelength, each 2D mask being generated by the same constant spatially coded aperture mask M*(i,j) with an appropriate wavelength dependent shift.

Let us assume a one-to-one correspondence between the [i,j] space at the detector where (i,j) ∈ [symbol] ^{N}^{ₓ}^{×N}^{_{y}} and the [x,y] space at the coded aperture where (x, y) ∈ [symbol] ^{N}^{ₓ}^{×N}^{_{y}}.

As an example, when the dispersing means causes a spatial shift d(Δλₙ) in the y direction (where Δλₙ is λₙ−λ_{c}, the spectral shift of the wavelength λₙ from a central wavelength λ_{c} then:

M(x, y+d(λₙ−λ_{c}), λₙ)=M*(x,y)

The 2D mask for each wavelength can be represented as a matrix {M⁽ⁿ^{_{λ)}}}ₙ_{_{λ}}₌₁^{N}^{_{λ}} ∈ [symbol] ^{N}^{ₓ}^{×N}^{_{y}}. This allows the measurement z obtained by each pixel 305 to be written in matrix form as

z=Hs, (4)

where z is a vectorized version of the measurement obtained by each pixel 305, s is the stacked vector of the three dimensional input beam of light S₀(x, y, λ) and H ∈ [symbol] ^{(N}^{ₓ}^{N}^{_{y}}^{)×(N}^{ₓ}^{N}^{_{y}}^{N}^{_{λ}}⁾ is the sensing matrix and can be represented by equation 5.

H=[Diag(M⁽¹⁾), . . . Diag(M^{(N}^{_{λ}}⁾)] (5)

In examples of the disclosure s is the spectral domain signal provided by an OCT arrangement. This allows equation (5) to be rewritten as

z=HFx (6)

Where x ∈ [symbol] ^{N}^{ₓ}^{N}^{_{y}}^{N}^{_{λ}} denote the three dimensional image of the object and F is the Fourier transform F ∈ [symbol] ^{(N}^{ₓ}^{N}^{_{y}}^{N}^{_{λ}}^{)×(N}^{ₓ}^{N}^{_{y}}^{N}^{_{λ}}⁾.

Therefore the measurement z₁ obtained by the first detecting means 109A and the measurement z₂ obtained by the second detecting means 109B can be written as:

z₁=H₁F₁x (7)

z₂=H₂F₂x (8)

The image can therefore be obtained by solving

$$x = arg {min}_{x} \frac{1}{2} {‖ {z}_{1} - {H}_{1} {F}_{1} x ‖}_{2}^{2} + β {‖ {z}_{2} - {H}_{2} {F}_{2} x ‖}_{2}^{2} + τ R ( x ) \qquad ( 9 )$$

Where R(x) denotes the regularizer imposed on the OCT image x, and τ and β balance the three terms in equations (9). For example, one choice for the value of β would be ½. Any suitable compressive sensing inversion algorithms may be used by processing means to solve equation (9) to obtain the desired image.

For example, processing means can use non-linear optimization to produce a three-dimensional image of the object. The processing means can be part of the apparatus 101 or separate from the apparatus 101. The processing means can comprise memory and a processor or controller. The non-linear optimization can, for example, minimize a cost function dependent upon all of the measurement channels. The cost function can be based on a summation, for each measurement channel, of a difference between a measurement and an expected measurement for that measurement channel.

In this example only two detectors 109 are provided. It is to be appreciated that a similar equation could be solved where the apparatus 101 comprises more than two channels and more than two detectors 109 are provided.

FIG. 4 illustrates an OCT arrangement 121 and an apparatus 101. In the example shown in FIG. 4 the apparatus 101 comprises two channels and the splitting means 103 splits the input beam of light 111 into a first beam 113A and a second beam 113B. It is to be appreciated that in other examples the apparatus 101 could comprise more than two channels and the splitting means 103 could be configured to split the input beam of light 11 into more than two beams.

The OCT Arrangement 121 comprises a light source 401, a beam splitter 403, a static reference mirror 405 and one or more focusing elements 407. The OCT arrangement 121 shown in FIG. 4 is a spectral domain arrangement.

In examples of the disclosure the light source 401 is a broadband light source which provides light having a range of wavelengths (frequencies). The wavelength of the light that is used may depend on the type of object 301 that is to be imaged or any other suitable factor. In some examples the light used may be infrared light. In some examples the wavelength of the light used may have a spectral bandwidth between 400 nm to 1500 nm.

The OCT arrangement 121 is configured so that the output light beam from the light source 401 is incident on the beam splitter 403. The beam splitter 403 may comprise a prism, a half silvered mirror or any other suitable component.

In the OCT arrangement 121 shown in FIG. 4 half of the split beam provides the reference beam and is provided to the static reference mirror 405. One or more focussing elements 407 are provided between the beam splitter 403 and the static reference mirror 405. The one or more focussing elements 407 may comprise any means which may be arranged to focus the beam of light. In some examples the one or more focussing elements 407 may comprise one or more lenses or any other suitable optical elements.

The other half of the split beam provides the object beam and is provided to the object 301. The object 301 may be arranged to be moved along the z axis. This axis may enable the focussing of the images provided by the OCT arrangement 121 and the apparatus 101. In the example of FIG. 4 the object 301 is provided on a motorised arrangement so as to enable movement along the z axis. In other examples a manual arrangement, or any other suitable type of arrangement, could be used.

One or more focussing elements 407 are provided between the beam splitter 403 and the object 301. The one or more focussing elements 407 may comprise any means which may be arranged to focus the beam of light. In some examples the one or more focussing elements 407 may comprise one or more lenses or any other suitable optical elements.

The different wavelengths of the light provide coherence of the object beam and the reference beam at different optical path lengths. Therefore the different wavelengths of light provide information about different depths within the object 301. Different features within the object 301 reflect the incident light by different amounts. The interference between the reflected object beam and the reflected reference beam therefore provides information about the features within the object 301.

As the different wavelengths of light provide information about different depths within the object 301 this enables three dimensional imaging of the object 301. The three dimensional imaging of the object 301 may enable different features at different depths within the object 301 to be identified and/or analysed. This ensures that the information obtained in the examples of the disclosure comprises information about the internal structure of an object 301 and not just information about the surface of the object 301.

The apparatus 101 is coupled to the OCT arrangement 121 so that the broadband output beam of light from the OCT arrangement 121 is provided as an input beam of light 111 to the apparatus 101.

The input beam of light 111 is provided to the splitting means 103 (a beam splitter component, for example, a half silvered mirror) so as to provide a first beam of light 113A and a second beam of light 113B. In this example and other examples, the spectral bandwidth of the first beam of light 113A and the spectral bandwidth of the second broadband beam of light 113B overlap and can be the same. The spectral bandwidth of the first beam of light 113A and the spectral bandwidth of the second broadband beam of light 113B are both broadband each covering multiple wavelength channels of a three-dimensional data cube [x, y, λ].

The first beam of light 113A is provided to a first channel. The first channel comprises a first modulating means 105A, a first dispersing means 107A and a first detecting means 109A.

In the first channel a focussing element 411 is provided between the splitting means 103 and the first modulating means 105A. The focussing element 411 may comprise one or more lenses or any other suitable means that may be configured to focus the first beam of light 113A onto the first modulating means 105A.

The first modulating means 105A provides a first modulated beam of light 115A. A focussing element 411 is provided between the first modulating means 105A and the first dispersing means 107A. The focussing element 411 may comprise one or more lenses or any other suitable means that may be configured to focus the first modulated beam of light 115A onto the first dispersing means 107A.

The first dispersing means 107A provides a first dispersed beam of light 117A. In the example shown in FIG. 4 the first dispersing means 107A may be configured to disperse the light in a horizontal direction. The horizontal direction could be parallel, or substantially parallel, with the x axis as shown in FIG. 4.

A focussing element 411 is provided between the first dispersing means 107A and the first detecting means 109A. The focussing element 411 may comprise one or more lenses or any other suitable means that may be configured to focus the first dispersed beam of light 117A onto the first detecting means 109A.

The second beam of light 113B is provided to a second channel. The second channel comprises a second modulating means 105B, a second dispersing means 107B and a second detecting means 109B.

In the second channel a focussing element 411 is provided between the splitting means 103 and the second modulating means 105B. The focussing element 411 may comprise one or more lenses or any other suitable means that may be configured to focus the second beam of light 113B onto the second modulating means 105B.

The second modulating means 105B provides a second modulated beam of light 115B. A focussing element 411 is provided between the second modulating means 105B and the second dispersing means 107B. The focussing element 411 may comprise one or more lenses or any other suitable means that may be configured to focus the second modulated beam of light 115B onto the second dispersing means 107B.

The second dispersing means 107B provides a second dispersed beam of light 117B. In the example shown in FIG. 4 the second dispersing means 107B may be configured to disperse the light in a vertical direction. The vertical direction could be parallel, or substantially parallel, with the y axis as shown in FIG. 4. The vertical direction could be perpendicular, or substantially perpendicular, with the horizontal direction in which the first dispersing means 107A disperses the light in the first channel.

A focussing element 411 is provided between the second dispersing means 107B and the second detecting means 109B. The focussing element 411 may comprise one or more lenses or any other suitable means that may be configured to focus the second dispersed beam of light 117B onto the second detecting means 109B.

The output signal from the first detecting means 109A and the output signal from the second detecting means 109B may be combined to provide a combined image. The combined image may comprise more information than can be provided by a single channel.

In the example shown in FIG. 4 the first dispersing means 107A in the first channel disperse light in a direction which is perpendicular, or substantially perpendicular to the direction that the second dispersing means 107B in the second channel disperse the light. It is to be appreciated that this does not need to be the case in all examples of the disclosure. In other examples the different directions do not need to be perpendicular to each other.

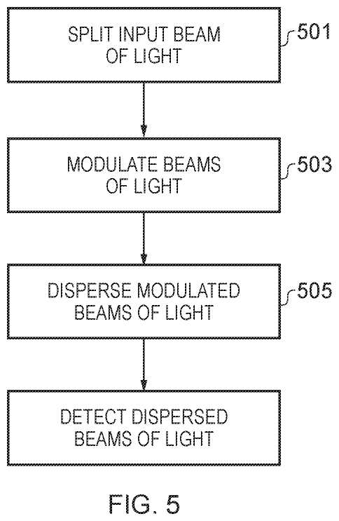

FIG. 5 illustrates an example method. The method may be implemented using any of the example apparatus 101 described above.

At block 501 the method comprises splitting an input beam of light 111. The input beam of light 111 is obtained from an OCT arrangement 121. The input beam of light 111 may be split into at least a first beam of light 113A and a second beam of light 113B.

At block 503 the method comprises modulating the beams of light 113A, 113B from the beam splitter. The first beam of light 113A is modulated to provide a first modulated beam of light 115A and the second beam of light 113B is modulated to provide a second modulated beam of light 115B.

At block 505 the method comprises dispersing the modulated beams of light 115A, 115B. The first modulated beam of light 115A is dispersed to provide a first dispersed beam of light 117A and the second modulated beam of light 115B is dispersed to provide a second dispersed beam of light 117B.

At block 507 the method comprises detecting the dispersed beams of light 117A, 117B and converting the detected beam of light into electrical output signals 119A, 119B.

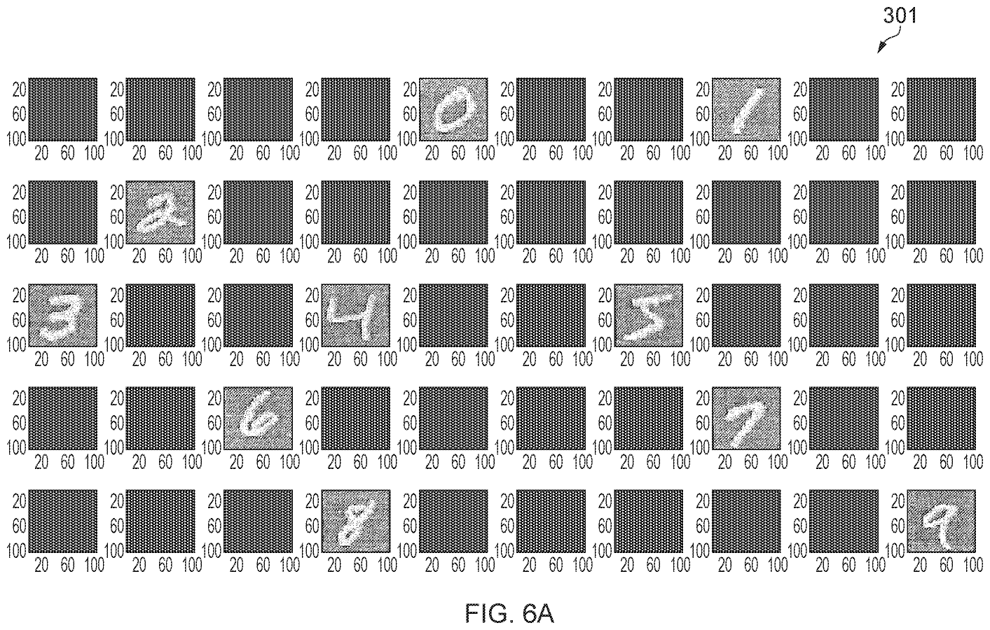

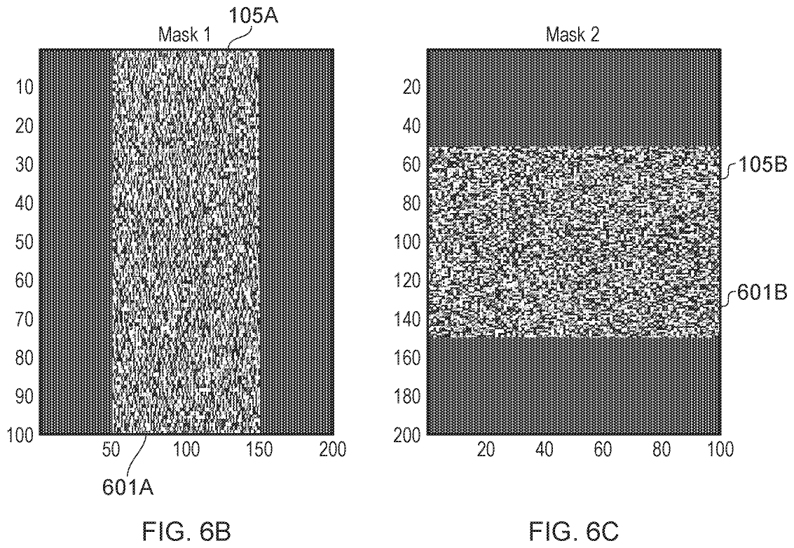

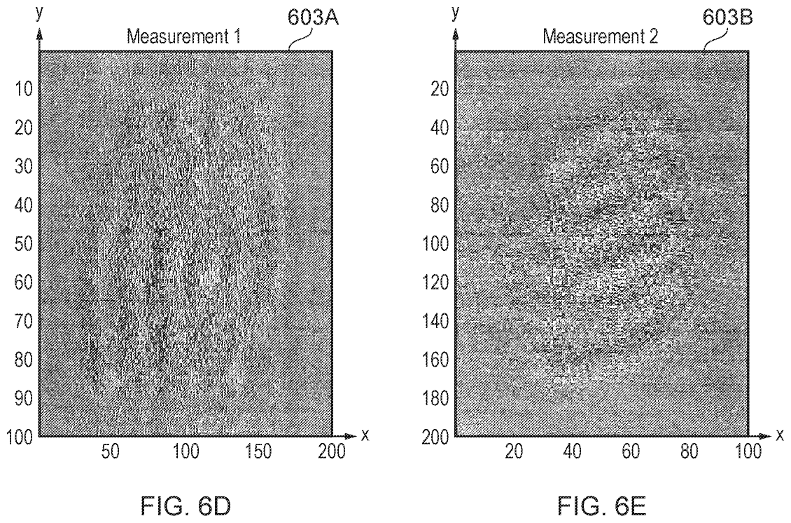

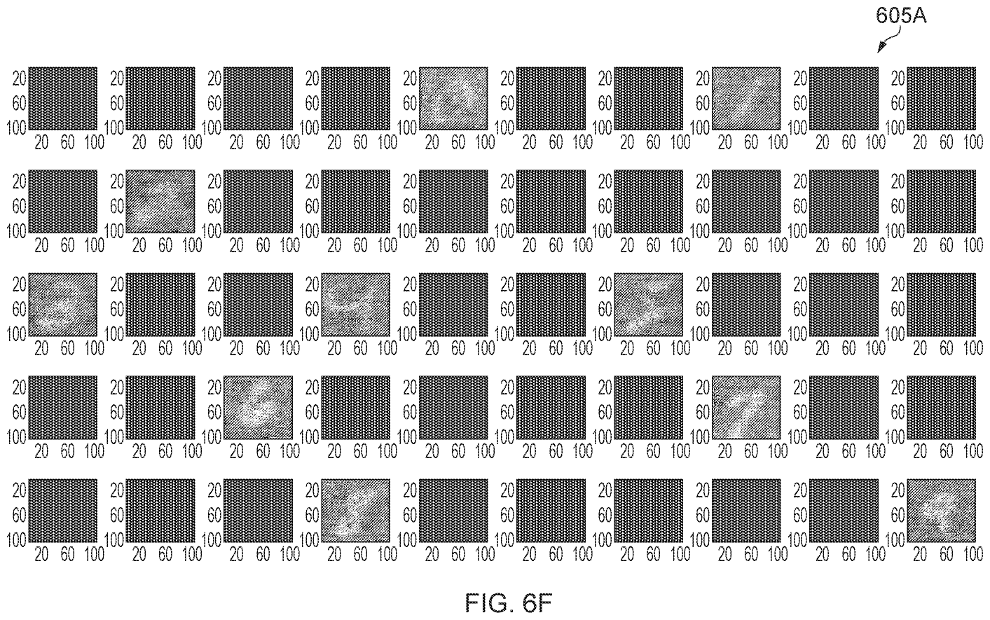

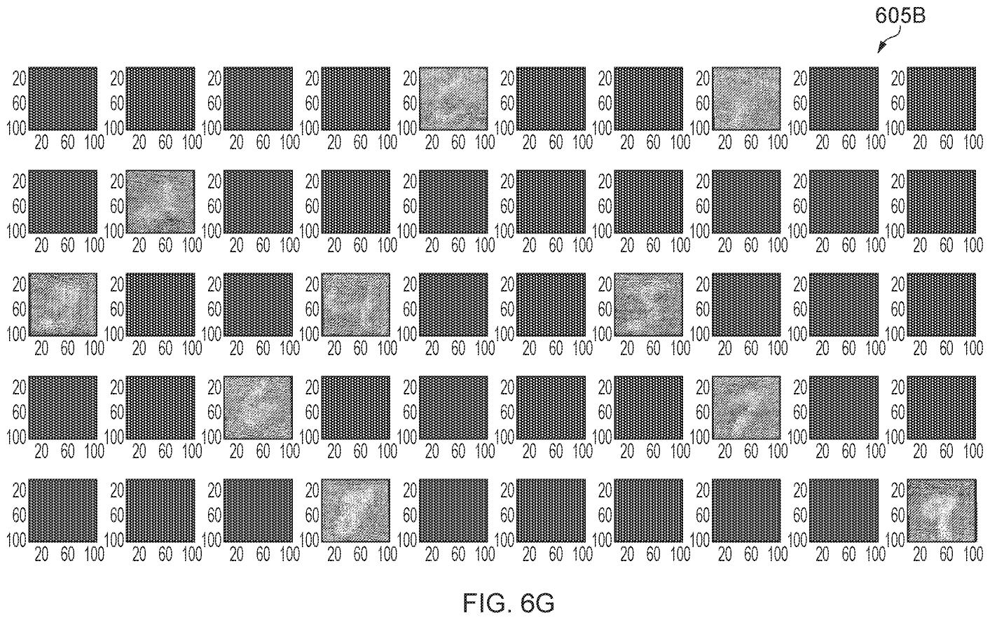

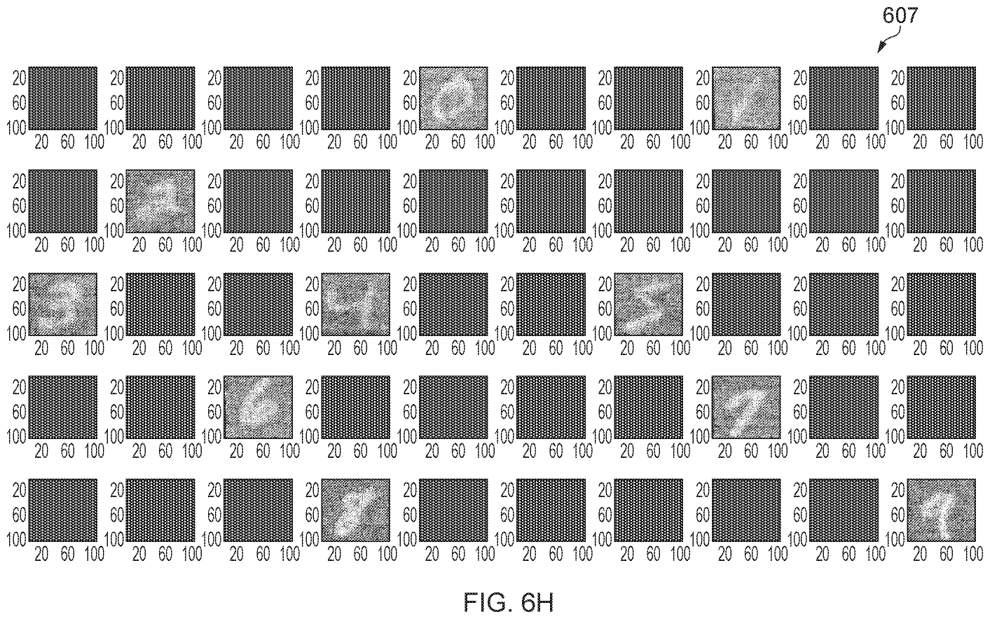

It is to be appreciated that in some examples the method may comprise further blocks that are not shown in FIG. 6. For instance, in some examples a modulating means 105 such as a coded aperture may be used to modulate the beams of light 113 and the method may comprise moving the modulating means so that different bandwidths are detected sequentially.

FIGS. 6A to 6H illustrate example images that may be obtained using an example apparatus 101. In the examples shown in FIGS. 6A to 6H the object 301 was imaged using a broadband light source with centre wavelength of 830 nm and a bandwidth of 20 nm. Other types of light sources could be used in other examples of the disclosure. In the examples shown in FIGS. 6A to 6H the images are obtained using an apparatus 101 having two channels. It is to be appreciated that apparatus 101 comprising more channels could be used in other examples of the disclosure.

FIG. 6A illustrates an example object 301 that is imaged by the OCT arrangement. In the example of FIG. 6A the object is a three dimensional image array with dimensions of 100×100×50. Ten of the depths have a number on them as shown in FIG. 6A. Each of the frames shown in FIG. 6A represents a different depth of the image array.

FIGS. 6B and 6C show masks 601A, 601B that can be used as modulating means 105A, 105B in examples of the disclosure. The first mask 601A may be provided as the first modulating means 105A in the first channel and the second mask 601 may be provided as the second modulating means 105B in the second channel.

In the examples shown in FIGS. 6B and 6C the masks are binary masks. Each pixel within the masks has a value of 0 or 1 where 0 blocks the light and 1 enables passing of the light. It is to be appreciated that in some examples greyscale masks could be used where some of the pixels may enable part of the light to pass through.

In the examples shown in FIGS. 6B and 6C the arrangement of the pixels in the masks 601, 601B is random or pseudo random. Other arrangements of the pixels could be used in other examples of the disclosure. The arrangement of the pixels in the masks 601A, 601B could be selected based on the types of objects 301 that are to be imaged or any other suitable factors. In such examples the pixels could be arranged in a customised pattern.

In the examples shown in FIGS. 6B and 6C the first mask 601A is the same as the second mask 601B. However the second mask 601B has been rotated through 90° so that the second mask is perpendicular or substantially perpendicular to the first mask 601A. It is to be appreciated that other arrangements of the masks 601A, 601B could be used in other examples of the disclosure.

FIGS. 6D and 6E show measurements that may be obtained by the detectors 109A, 109B of the apparatus 101. FIG. 6D shows an example measurement 603A that may be obtained by the first detector 109A in the first channel and FIG. 6E shows an example measurement 603B that may be obtained by the second detector 109B in the second channel. In the first channel the dispersing means 107A has been configured to spread the light in a horizontal, or substantially horizontal, direction as indicated by the x axis in FIG. 6D. In the second channel the dispersing means 107B has been configured to spread the light in a vertical, or substantially vertical, direction as indicated by the y axis in FIG. 6E.

FIG. 6F shows a reconstructed image 605A of the object 301 that is obtained from the measurement obtained by the first detector 109A. The reconstructed image 605A may be obtained using the methods described above in relation to FIG. 3 or any other suitable method. Each of the frames shown in FIG. 6F show a different depth of the array of the object 301. In the example shown in FIG. 6F the peak-signal-to noise-ratio (PSNR) compared with truth is 23.1370 dB.

FIG. 6G shows a reconstructed image 605B of the object 301 that is obtained from the measurement obtained by the second detector 109B. The reconstructed image 605B may be obtained using the methods described above in relation to FIG. 3 or any other suitable method. Each of the frames shown in FIG. 6G show a different depth of the array of the object 301. In the example shown in FIG. 6G the PSNR compared with truth is 23.0169 dB.

FIG. 6H shows a reconstructed image 607 of the object 301 that is obtained from the both the measurements obtained by first detector 109A and the measurements obtained by the second detector 109B. The reconstructed image 6057 may be obtained by solving equation (9) as described above in relation to FIG. 3 or any other suitable method. Each of the frames shown in FIG. 6H show a different depth of the array of the object 301. In the example shown in FIG. 6H the PSNR compared with truth is 26.4347 dB. Therefore the reconstructed image 607 that is obtained by combining measurements from two or more channels has a higher PSNR than the images obtained from single channels. Therefore the multi-channel apparatus 101 enables a higher quality image 607 to be obtained from the OCT arrangement 121.

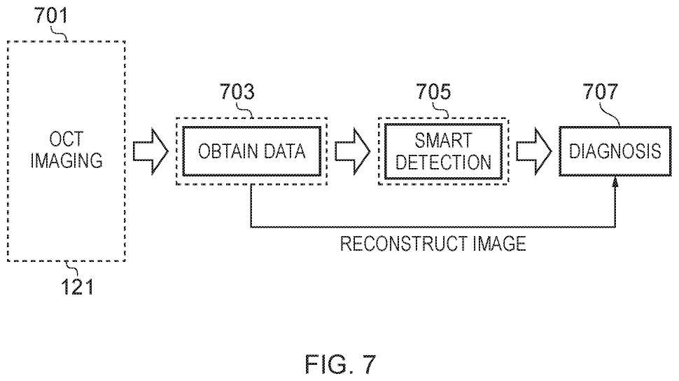

FIG. 7 illustrates a method of using examples of the disclosure which may be used to enable a medical diagnosis. At block 710 the OCT imaging is performed. The object 301 that is imaged may the retina of a person or animal or any other suitable object 301. The OCT imaging is performed by an OCT arrangement 121 which may be as described above.

At block 703 the data from the OCT imaging is obtained. The data is obtained by an apparatus 101 or plurality of apparatus 101 as described above. The apparatus 101 could comprise two or more different channels as described above. This enables a higher quality image to be obtained with a higher PSNR than an image obtained using a single channel.

The data obtained by the apparatus 101 or plurality of apparatus 101 is modulated by the modulating means 105. This enables the data to be captured in a compressed manner. This may provide for an efficient use of memory circuitry and communication bandwidths.

The obtained data is detected by the detecting means 109 as described above. The electrical output of the detecting means 109 may be used, at block 705 for smart detection. The smart detection may comprise the use of algorithms, or any other suitable technique, to recognise features in the output signal of the detecting means 109. This information could then be provided to the user, who may be a medical professional, at block 7077. The information that is provided may be used to enable a diagnosis by the medical professional.

In some examples, at block 707, the output signal from the detecting means 109 may be used to reconstruct an image of the object 301, or at least part of the object 301. This may also be provided to the medical professional to assist with any diagnosis.

The described examples therefore provide apparatus 101 comprising a plurality of channels which enable images with improved PSNRs to be obtained. The example apparatus 101 also provide for a fast capture of the information while providing for efficient use of communications bandwidths and memory circuitry.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to ‘comprising only one . . . ’ or by using ‘consisting’.

In this description, reference has been made to various examples. The description of features or functions in relation to an example indicates that those features or functions are present in that example. The use of the term ‘example’ or ‘for example’ or ‘can’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’, ‘can’ or ‘may’ refers to a particular instance in a class of examples. A property of the instance can be a property of only that instance or a property of the class or a property of a sub-class of the class that includes some but not all of the instances in the class. It is therefore implicitly disclosed that a feature described with reference to one example but not with reference to another example, can where possible be used in that other example as part of a working combination but does not necessarily have to be used in that other example.

Although embodiments have been described in the preceding paragraphs with reference to various examples, it should be appreciated that modifications to the examples given can be made without departing from the scope of the claims.

Features described in the preceding description may be used in combinations other than the combinations explicitly described above.

Although functions have been described with reference to certain features, those functions may be performable by other features whether described or not.

Although features have been described with reference to certain embodiments, those features may also be present in other embodiments whether described or not.

The term ‘a’ or ‘the’ is used in this document with an inclusive not an exclusive meaning. That is any reference to X comprising a/the Y indicates that X may comprise only one Y or may comprise more than one Y unless the context clearly indicates the contrary. If it is intended to use ‘a’ or ‘the’ with an exclusive meaning then it will be made clear in the context. In some circumstances the use of ‘at least one’ or ‘one or more’ may be used to emphasis an inclusive meaning but the absence of these terms should not be taken to infer and exclusive meaning.

The presence of a feature (or combination of features) in a claim is a reference to that feature or (combination of features) itself and also to features that achieve substantially the same technical effect (equivalent features). The equivalent features include, for example, features that are variants and achieve substantially the same result in substantially the same way. The equivalent features include, for example, features that perform substantially the same function, in substantially the same way to achieve substantially the same result.

In this description, reference has been made to various examples using adjectives or adjectival phrases to describe characteristics of the examples. Such a description of a characteristic in relation to an example indicates that the characteristic is present in some examples exactly as described and is present in other examples substantially as described.

Whilst endeavoring in the foregoing specification to draw attention to those features believed to be of importance it should be understood that the Applicant may seek protection via the claims in respect of any patentable feature or combination of features hereinbefore referred to and/or shown in the drawings whether or not emphasis has been placed thereon.

1-15. (canceled)

16. An apparatus comprising:
a beam splitter configured to split an input beam of light into at least a first beam of light and a second beam of light wherein the input beam of light is obtained from an optical coherence tomography arrangement and wherein the spectral bandwidth of the first beam of light and the spectral bandwidth of the second beam of light overlap;
one or more spatial modulators configured for spatially modulating the first beam of light to provide a first spatially modulated beam of light and spatially modulating the second beam of light to provide a second spatially modulated beam of light;
one or more dispersers configured to disperse, in a first direction, the first spatially modulated beam of light to provide a first dispersed beam of light and to disperse, in a second direction different from the first direction, the second spatially modulated beam of light to provide a second dispersed beam of light; and
one or more detectors configured to detect the first dispersed beam of light and the second dispersed beam of light, wherein the one or more detectors are configured to convert the detected beams of light into electrical output signals.

17. The apparatus of claim 16, wherein the beam splitter is configured to split the input beam of light into more than two beams of light.

18. The apparatus of claim 17, further comprising:
a first spatial modulator configured for spatially modulating the first beam of light;
a first disperser configured for dispersing the first spatially modulated beam of light;
a first detector configured for detecting the first dispersed beam of light;
a second spatial modulator configured for spatially modulating the second beam of light;
a second disperser configured for dispersing the second spatially modulated beam of light; and
a second detector configured for detecting the second dispersed beam of light.

19. The apparatus of claim 16, wherein at least one of the one or more second spatial modulators comprises one or more coded apertures.

20. The apparatus of claim 19, wherein the one or more coded apertures comprise a two dimensional pixelated coded aperture.

21. The apparatus of claim 19, wherein the one or more coded apertures comprise at least first portions having a first transparency to the beam of light and at least second portions having a second transparency to the beam of light, the second transparency being different from the first transparency.

22. The apparatus of claim 21, wherein the first transparency and the second transparency are wavelength dependent.

23. The apparatus of claim 21, wherein the first and second portions of the one or more coded apertures are arranged in a random pattern.

24. The apparatus of claim 16, wherein the one or more spatial modulators are arranged to be moveable relative to the one or more dispersers and the one or more detectors.

25. The apparatus of claim 16, wherein the one or more spatial modulators comprise at least a first plurality of first portions having a first transparency to an input beam of light and at least a second plurality of second portions having a different transparency to the input beam of light, wherein the first and second portions of the one or more spatial modulators are arranged in a pixelated arrangement.

26. The apparatus of claim 16, wherein the one or more dispersers comprise at least one of: a prism or a grating.

27. The apparatus of claim 16, wherein the one or more detectors comprise a two dimensional array of sensors.

28. The apparatus of claim 16, wherein the optical coherence tomography arrangement is arranged so that the input beam of light comprises different wavelengths of light and the different wavelengths of light provide information about different depths within an object.

29. The apparatus of claim 28, further comprising:
one or more processors configured for processing the electrical output signals and causing generation of a three dimensional image of at least part of the object.

30. A method comprising:
splitting an input beam of light into at least a first beam of light and a second beam of light wherein the input beam of light is obtained from an optical coherence tomography arrangement and wherein the spectral bandwidth of the first beam of light and the spectral bandwidth of the second beam of light overlap;
spatially modulating the first beam of light to provide a first spatially modulated beam of light and spatially modulating the second beam of light to provide a second spatially modulated beam of light;
dispersing the first spatially modulated beam of light, in a first direction, to provide a first dispersed beam of light and dispersing the second spatially modulated beam of light, in a second direction different to the first direction, to provide a second dispersed beam of light; and
detecting the first dispersed beam of light and detecting the second dispersed beam of light and converting the detected beams of light into electrical output signals.

31. The method of claim 30, wherein said splitting the input beam of light comprises splitting the input beam of light into more than two beams of light.

32. The method of claim 30, wherein said spatially modulating is carried out using at least one or more spatial modulators comprising one or more coded apertures.

33. The method of claim 32, wherein the one or more coded apertures comprise at least first portions having a first transparency and at least second portions having a second transparency to the beam of light, the second transparency being different from the first transparency, wherein the first transparency and the second transparency are wavelength dependent.

34. The method of claim 30, wherein the optical coherence tomography arrangement is arranged so that the input beam of light comprises different wavelengths of light and the different wavelengths of light provide information about different depths within an object.

35. The method of claim 34, further comprising:
providing a three dimensional image of at least part of the object based at least upon the electrical output signals.
